(19) United States
(12) Patent Application Publication
(10) Pub. No.: US 2021/0290962 A1
(43) Pub. Date: Sep. 23, 2021

(54) CONTROL CIRCUIT FOR IMPLANTABLE PULSE GENERATOR

(71) Applicant: ONWARD MEDICAL B.V., Eindhoven (NL)

(72) Inventors: Koen WEIJAND, Eindhoven (NL); Jeroen TOL, Eindhoven (NL)

(73) Assignee: ONWARD MEDICAL B.V., Eindhoven (NL)

(21) Appl. No.: 17/206,030

(22) Filed: Mar. 18, 2021

(30) Foreign Application Priority Data
Mar. 19, 2020 (EP) 20164082.8

(51) Int. Cl. A61N 1/36; A61N 1/02; G16H 20/30

(52) U.S. Cl. CPC A61N 1/36153; G16H 20/30; A61N 1/36157; A61N 1/025

(57) ABSTRACT

Systems and methods are disclosed for managing a tissue node potential of a neurostimulation system. The envisioned neurostimulation system can include an energy source, a housing or a housing portion, a stimulation node, a stimulation surface for contacting a tissue of a patient, and a control circuit. The control circuit can be configured to maintain at least one of the housing or housing portion, the stimulation node, or the stimulation surface at a predefined voltage or within a predefined voltage range.

### CROSS-REFERENCE TO RELATED APPLICATION

This application claims the benefit of priority of European Patent Application No. EP20164082.8, filed Mar. 19, 2020, titled “CONTROL CIRCUIT FOR IMPLANTABLE PULSE GENERATOR,” the entire disclosure of which is incorporated herein by reference.

### BACKGROUND

A power control loop for implantable tissue stimulators can be established between an external control device and an implantable device so that only the amount of power needed by the implant device to sustain its present operating conditions is transmitted across a transcutaneous transmission link, thereby reducing the amount of power expended by the external control device.

There can be a control of externally induced current in an implantable pulse generator. A current-limiting apparatus and method for limiting current flow can be induced when the level of an external signal is greater than an external signal threshold signal level, in a conductive loop formed by a medical device implanted within a living organism having electrically excitable tissue. Such a control can be provided with an implantable pulse generator (IPG) system having a housing, a signal generator disposed in the housing that generates an electrical signal, and at least one lead extending from the housing to convey electrical signal to the patient. To limit the induced current flow, the IPG can include current limiting componentry, an impedance increasing element, and/or alternating current blocking elements. These components can provide an alternating current impedance path to the electrical ground from a lead coupled to the capacitive element. There are techniques for reducing the effective surface area of the current inducing loop caused by the IPG system.

An amount of current provided in the electrical stimulation of tissue can be controlled. A charge setting can be provided for specifying an amount of charge that is to flow during a stimulation pulse that electrically stimulates a tissue. The stimulation pulse can then be generated and delivered the stimulation pulse in a manner such that an amount of charge delivered to the tissue during the stimulation pulse accords with the charge setting.

### BRIEF DESCRIPTION OF THE DRAWINGS

The accompanying drawings, which comprise a part of this specification, illustrate several embodiments and, together with the description, serve to explain the principles and features of the disclosed embodiments. In the drawings:

FIG. 1A depicts a generalized node voltage controller in a form of a regulated CAN controller, forming an embodiment of a control circuit according to the present invention, with which the method according to the present invention may be performed;

FIG. 1B depicts a control circuit according to the present invention, with which the method according to the present invention may be performed.

FIG. 1C depicts an exemplary method for regulating a potential of a stimulation node, consistent with disclosed embodiments.

FIG. 2A depicts a further schematic illustration of a control circuit according to the present invention according to FIG. 1B, with which the method according to the present invention may be performed.

FIG. 2B depicts a control circuit according to the present invention, with which the method according to the present invention may be performed.

FIG. 3 depicts a control circuit according to the present invention, with which the method according to the present invention may be performed.

FIG. 4 depicts a control circuit according to the present invention, with which the method according to the present invention may be performed.

FIG. 5 depicts a control circuit according to the present invention, expanded with a second monitoring element, with which the method according to the present invention may be performed.

### DETAILED DESCRIPTIONS

Reference will now be made in detail to exemplary embodiments, discussed with regards to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

Differences in potential between a tissue node (e.g., an electrode, a stimulator housing, or the like) and tissue can result in corrosion and tissue damage. Certain conventional systems use dual-supply rails to manage such potential differences. However, such conventional systems can require additional space and power. As described herein, charge-balanced stimulation can enable management of such potential differences using a capacitor without requiring a dedicated supply rail.

Consistent with disclosed embodiments, the potential of a tissue node of a stimulation engine can be managed in a more power-efficient and compact manner than implementations using a dedicated supply rail to control the stimulation node's potential (e.g., through a boost converter or an LDO type of circuit). The tissue node of a stimulation engine can be any node and/or surface configured for delivering and/or controlling stimulation to tissue. Typical examples of such stimulation nodes or surfaces can be the housing or housing portion of a neurostimulator, an electrode of a lead, or any other stimulation node or stimulation surface configured for delivering and/or controlling stimulation to tissue.

Consistent with disclosed embodiments, such a tissue potential can be managed using a control circuit for an implantable pulse generator with the features of claim 1. Accordingly, a control circuit for a neurostimulator, especially for an implantable pulse generator for a neurostimulator, comprising and/or being connected to at least one energy source, at least one housing or housing portion and/or at least one stimulation node and/or at least one stimulation surface configured to be in contact with tissue of a patient, at least one capacitor being directly or indirectly connected with the housing or housing portion and/or the stimulation node and/or the stimulation surface, the control circuit and/or the capacitor being arranged and configured such that the housing or housing portion and/or the stimulation node and/or the stimulation surface can be kept at a predefined voltage or kept within a predefined voltage range by means of the control circuit and/or the capacitor.

Consistent with disclosed embodiments, a simple electronic element (e.g., a capacitor or other circuit element with a similar function) can be used to maintain the housing or at least a housing portion of an IPG (or any other implantable medical unit (IMU)) at a predefined voltage (or within a predefined voltage range). The housing may be for example the can (also CAN) of the implantable pulse generator. In this manner, a desired potential between tissue surrounding and/or in galvanic contact with the CAN can be maintained.

Accordingly, compact and efficient systems and methods for controlling the CAN potential of a neurostimulator are provided. Such systems and methods can set the tissue potential if the CAN is in direct (galvanic) contact with tissue, for example in case of an implantable pulse generator (IPG) for a neurostimulation/neuromodulation system. Such systems and methods may exhibit advantages over conventional approaches that use a dedicated supply for maintaining the potential of a CAN or CAN portion of a neurostimulator:

The control circuit may enable a single high-voltage supply-rail implementation of the stimulation engine (also STIM engine). This leads to a much-reduced footprint in comparison to a dual-rail supply implementation with both a positive and negative supply rail and CAN connected to ground (GND) (e.g. negative polarity of the battery). This is especially important for an IPG realization with discrete and off-the-shelf integrated circuits (ICs)where a dual-rail solution may have a prohibitively large area and volume.

In some implementations, controlling the CAN potential through a dedicated DC-DC converter (e.g. a boost converter or LDO type of circuit) may provide a power penalty, and consequently, a negative impact on battery longevity. For example, for stimulation with perfectly balanced stimulation pulses, ideally, there is no net current flow through the housing (averaged over a stimulation period) and the housing voltage is unchanged after each stimulation pulse. Consequently, there is no need for a DC-DC converter to keep the housing at a desired voltage which saves the otherwise consumed quiescent power consumption of such a converter. More realistically, even if the stimulation pulses have some charge imbalance, the resulting (average) current delivered by a dedicated DC-DC converter will be small in practice, and therefore, the resulting power efficiency of this converter will be poor.

Moreover, typically, a DC-DC converter can only source current which means that if the housing sinks more charge than it sources over a stimulation period, which cannot be excluded beforehand, then in practice, the reservoir output capacitor of the DC-DC converter needs to be regulated or controlled to ensure that the output voltage of the converter does not run away. As shown in this disclosure, this CAN controller can be implemented in such a way that the housing can both sink and source current without the need for a dedicated DC-DC converter at all.

The envisioned embodiments can benefit from a reduced power penalty. Consistent with disclosed embodiments, additional power consumption by the CAN controller may be driven by opening and closing switches, which may require very little power. Furthermore, a well-defined tissue potential through the CAN controller can improve stimulation pulse characteristics (e.g., pulse shape, amplitude, or the like) by ensuring that current sources of the STIM engine output stages remain in their high-ohmic compliance region during stimulation.

A single-rail STIM engine without controlling the CAN/tissue potential may consume twice the power to generate biphasic stimulation pulses than a pure dual-rail implementation. This doubling of the power consumption may be an issue with the limited available battery capacity that can be realized in an implant. However, a single-rail design with the CAN controller as possible in a preferred embodiment can be almost as power efficient as a dual-rail design but without the (prohibitively) large board area and implant volume claim.

As may be appreciated, stimulation can be charge-neutral in nature. As such, the net current flow through each branch (e.g. a wire) of a stimulated network (e.g. electrodes and CAN in tissue) may be zero (no DC). Or put differently, the sum of the currents over time through a node of interest (e.g. electrode or CAN) may be zero, containing no DC component (as otherwise electrode corrosion and/or tissue damage could occur for those nodes in contact with tissue). In applications where the amount of charge flow is also limited, as is the case for neurostimulation pulses (as a stimulation pulse contains a finite amount of charge) a node of interest can be kept at a desired voltage using a capacitor by monitoring and controlling the voltage of the capacitor.

The housing can be, in an example embodiment, the housing (portion) of a neurostimulator or a component of a neurostimulator. In particular, it can be the housing (portion) or CAN of an implantable pulse generator (IPG).

Consistent with disclosed embodiments, a potential of the housing or CAN of an IPG can be managed so that the CAN acts as a reference potential for the tissue (e.g., assuming the CAN is in direct contact with the tissue). However, any stimulation node or stimulation surface configured for delivering and/or controlling stimulation, and characterized by substantially net zero charge injection, can be used to apply this idea. Therefore, the wording ‘housing’, ‘housing portion’, ‘can’ or ‘CAN’ may freely be substituted for ‘stimulation node’ or ‘stimulation surface’ and likewise, the wording ‘CAN controller’, ‘can controller’ or simply ‘controller’ can be replaced with ‘stimulation node controller’ or ‘stimulation surface controller’.

The energy source can be a current source and/or a voltage source and/or power source. The energy source can be a battery or DC voltage source. Furthermore, the energy source can be connected directly and/or indirectly with the control circuit. Also, the energy source can be a part of the control circuit or can be separated from it.

The CAN portion may be configured to be in contact with tissue (e.g., implanted into a human body). The capacitor may be connected with the CAN portion (directly and/or indirectly). Furthermore, the CAN portion can be kept at a desired predefined voltage or (electric) potential by means of the capacitor, such that the housing or housing portion and thus also the surrounding tissue being in contact with the CAN and/or CAN portion can be kept at a desired predefined voltage or (electric) potential by means of the capacitor.

Further, the control circuit can comprise at least one energy controlling element, which can be configured such that it uses energy from or returns energy to the energy source to keep the housing and/or the housing portion and/or the stimulation node and/or the stimulation surface at a predefined voltage or kept within a predefined voltage range.

In other words, in an example embodiment, the control circuit can comprise at least one energy controlling element, which can be configured such that it controls the energy taken from the energy source to keep the housing and/or housing portion and/or the stimulation node and/or the stimulation surface at a predefined voltage or keep it within a predefined voltage range. By this, it is assured that by using the energy source itself, the voltage or (electric) potential of the housing or housing portion can be controlled and/or adjusted such that it stays at a predefined voltage or can be kept within a predefined voltage range.

In some embodiments, the energy controlling element can be configured such that charging the capacitor can draw energy from the energy source and discharging the capacitor can provide energy to the energy source. Accordingly, energy from the energy source can be used and funneled by the controlling element to control and/or adjust the voltage of the housing (portion) and/or the stimulation node and/or the stimulation surface and/or to supply the energy the housing and/or housing portion and/or the stimulation node and/or the stimulation surface delivers, for example, to tissue such that the housing and/or housing portion and/or the stimulation node and/or the stimulation surface is kept at a predefined voltage or kept within a predefined voltage range.

The energy controlling element can be embodied or its function can be provided by any electronic component being capable to do so. For example, the energy controlling element can be embodied by a (micro)controller or its functionality may be provided by an (already present) (micro)controller.

Furthermore, it is possible that the control circuit further comprises a monitoring element. In particular, the monitoring element can be configured such that by (or using) the monitoring element the voltage across the capacitor and/or the voltage of the housing and/or housing portion and/or the voltage of the stimulation node and/or the stimulation surface is monitored and/or the current through the capacitor and/or the housing and/or the housing portion and/or the stimulation node and/or the stimulation surface is monitored and/or the voltage and/or the current and/or the power of the energy source is monitored.

The monitoring element is an advantageous additional element, which assists the energy controlling element in its function. As such, the monitoring element monitors the voltage across the capacitor and/or the voltage or (electric) potential of the housing (portion) in order to control or correct the voltage across the capacitor or housing (portion).

By this, a monitoring can be done by the controlling element, especially in real-time. In particular, by this it is possible to monitor the at least one energy source and/or the at least one capacitor and/or the at least one housing (portion). The monitoring can include, but is not limited to, a monitoring of a current and/or a voltage and/or a charge. A more accurate monitoring of the control circuit is possible in this way. The monitoring element can make use of e.g. thresholds where staying within the thresholds is tolerated and no action is initiated and exceeding one or more thresholds will lead to an automatic, semi-automatic or manual correction.

Additionally, it is possible that the voltage across the at least one capacitor can be controlled and/or corrected by the energy controlling element with input from the monitoring element. The level of the at least one energy source and/or the voltage across the at least one capacitor and/or the voltage or (electric) potential of the at least one housing (portion) can be thus monitored and/or controlled and/or corrected by (or using) the energy controlling element with input from the monitoring element.

For example, the monitoring element can be embodied as a sensor, while the control circuit may comprise current sources configured in an H-bridge constellation as an actuator to control the level of the energy source and/or the voltage across the capacitor and/or the voltage of the housing (portion).

Especially, when the control circuit needs to deal with charge imbalanced pulses, monitoring is recommended. The reason for this is the fact that monophasic but also imperfect biphasic pulses are not charge neutral, and therefore can induce some drifting away in time, for example, of the voltage across the at least one capacitor. By doing the monitoring, such drifting away can easily be detected and corrected, when necessary or desired.

Furthermore, the voltage provided by the energy source may be a DC voltage. Such voltage can be easily provided by usual batteries with the batteries acting as an example energy source.

Furthermore, it is possible that the control circuit for the implantable pulse generator can be configured and arranged for handling bi-phasic pulses. By this, a broader range of neurostimulation can be provided and is possible.

Additionally, the control circuit may comprise at least one of a current source, a switch, a voltage supply, a ground, and a bridge circuit.

The housing and/or housing portion and/or the stimulation node and/or the stimulation surface can be part of the bridge circuit. Furthermore, the bridge circuit may comprise or be an H-bridge circuit.

Additionally, it is possible that a supply voltage may be within a range from 0 V to approx. 50 V, especially approx. 5 V to approx. 30 V, preferably from approx. 20 V to approx. 30V, and may be supplied either directly or indirectly by the energy source, for example, a voltage supply rail.

Further, the control circuit can comprise at least a capacitor, a monitoring element, a switch being arranged in a branch connecting the capacitor with a voltage supply and/or another voltage source, and a further switch being arranged in a branch connecting the capacitor with another rail.

Moreover, the control circuit can comprise at least one further current source.

Moreover, the control circuit can further comprise a first control switch, and a second control switch connected in series, wherein the first control switch and the second control switch are arranged between the first current source and the second current source.

Also, it is possible that the control circuit may comprise a first current source , a second current source, a first control switch, and a second control switch connected in series, wherein the first control switch and the second control switch are arranged between the first current source and the second current source.

The control circuit further may be arranged and provided such that it comprises a further switch for connecting the capacitor and/or the housing or housing portion to ground and/or another rail.

Furthermore, it is possible that the control circuit can be connected either directly or indirectly (e.g. via tissue) to a stimulation generation module of an implantable pulse generator, wherein the stimulation generation module can be powered from a single energy source.

Also it is possible that if the third control switch is in its open state after the capacitor has been charged or discharged to the defined/desired capacitor voltage by the first current and/or second current source, at least one monophasic or biphasic stimulation pulse can be generated by the stimulation generation module such that the capacitor can be further charged or discharged leading to a drifting away from the defined/desired capacitor voltage after the pulse has been output.

In a further embodiment it is possible that the bridge circuit may comprise a monitoring element for monitoring the capacitor voltage with a first resistor and a second resistor, the monitoring element can be connected to the first branch between the first current source and the second current source and can be further connected to the ground.

The monitoring element may also monitor the level of the energy source, for example, the control circuit may comprise a voltage source whose voltage level may be monitored by the monitoring element.

Additionally, it is possible that if the first and second control switch are in their open state and the third control switch is in its closed state, a difference between the actual capacitor voltage and the defined/desired capacitor voltage can be monitored by the monitoring branch of the first resistor and the second resistor, such that based on the difference, the first control switch or the second control switch can be closed.

Furthermore, it is possible that the control circuit may be connected either directly or indirectly, for example via tissue, to a stimulation generation module of the implantable pulse generator, wherein the stimulation generation module uses a single voltage source as energy source.

Furthermore, the voltage of the housing (portion) can be monitored and controlled/set with the goal to keep at least one stimulation current source of the stimulation generation module within its compliance range during stimulation.

Also, due to the (electric) potential and/or voltage being controlled and/or set at the housing or housing portion and/or the stimulation node and/or the stimulation surface configured to be in contact with tissue of a patient, at least one stimulation current source or output stage of the stimulation generation module remains in its high-ohmic output range and/or its compliance region during stimulation. If the output stage is voltage driven, instead of current driven via a current source, the control circuit can maintain the desired/defined voltage between the inherent voltage source of the at least one voltage driven output stage and the housing (portion) in contact with tissue.

Furthermore, in conjunction with the above-described circuit, the following method of controlling a neuromodulation and/or neurostimulation system, especially an implantable pulse generator for a neurostimulation and/or neurostimulation system is disclosed. Accordingly, a method for controlling an implantable pulse generator is provided, comprising the following steps: providing at least one energy source, providing at least one housing or housing portion configured to be in contact with tissue of a patient, providing at least one capacitor being directly or indirectly connected with the housing or housing portion, and keeping the housing or housing portion at a desired predefined voltage or within a predefined voltage range by (or using) the capacitor.

The method may be further arranged and conducted such that it comprises the following steps: providing the control circuit for the implantable pulse generator comprising the bridge circuit including the capacitor, wherein the capacitor and/or the bridge circuit being connected to the outer housing (portion) of the implantable pulse generator staying in direct contact with tissue; and monitoring and/or controlling the voltage of the capacitor by the bridge circuit such that the housing voltage of the outer housing (portion) can be directly or indirectly set by the capacitor voltage.

It is possible that the housing voltage is kept at least one defined housing voltage (or within a predefined voltage range) if the capacitor voltage is also kept at least one defined capacitor voltage (or predefined voltage range), wherein the housing voltage and the capacitor voltage are different from or substantially equally to each other.

Additionally, it is possible that after the capacitor has been charged or discharged to the defined/desired capacitor voltage that at least one stimulation pulse is generated such that the capacitor can be further charged or discharged after the stimulation pulse has been output. This arising difference between the actual capacitor voltage and the defined/desired capacitor voltage may be monitored such that in response to that difference the capacitor can be charged or discharged to bring the capacitor voltage back to or near the defined/desired capacitor voltage.

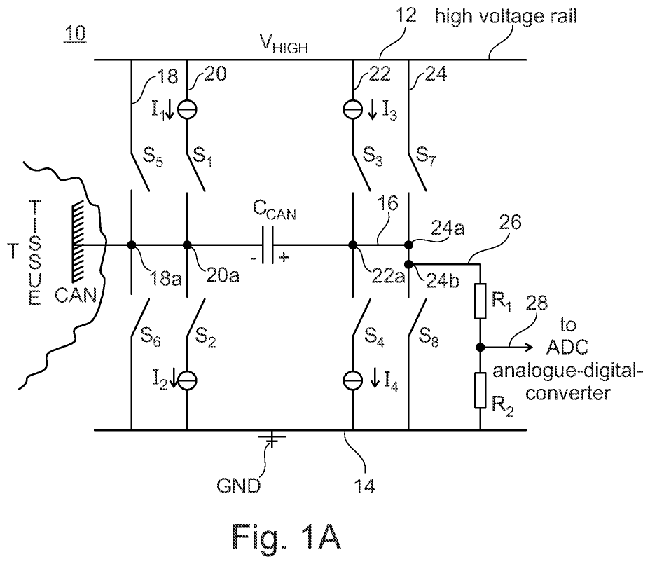

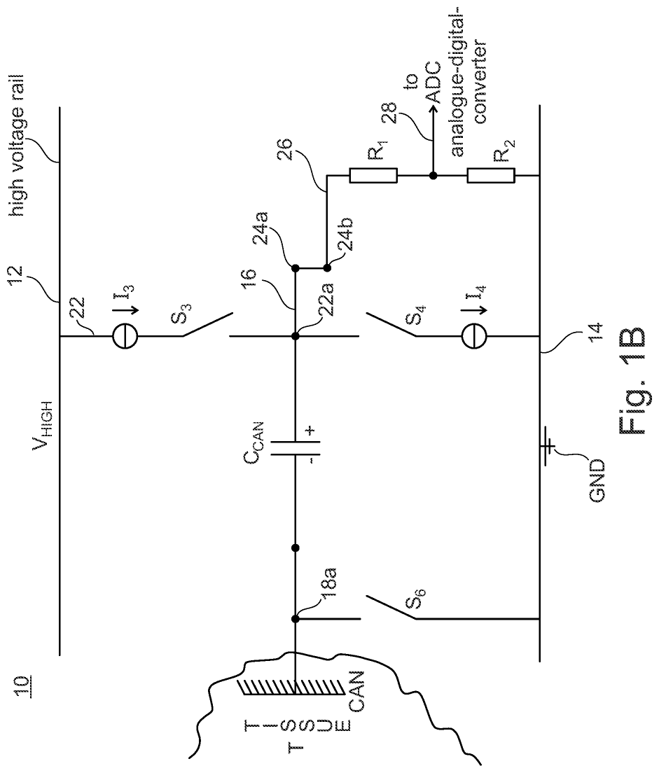

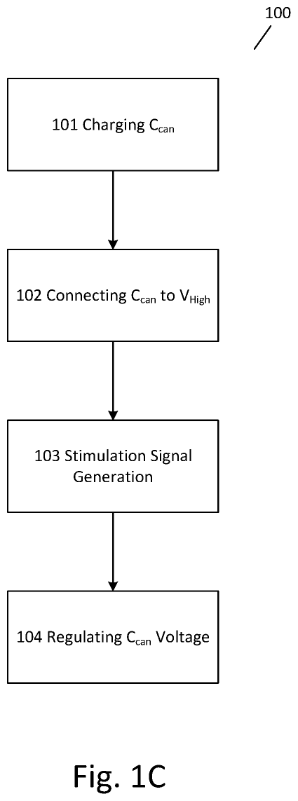

FIG. 1A shows a generalized node voltage controller in a form of a regulated CAN controller, forming an embodiment of a control circuit 10 according to the present invention, with which the method according to the present invention may be performed.

The control circuit 10 comprises a high voltage rail 12.

Furthermore, there can be another rail 14 on the other side, which is connected to ground (GND).

Additionally, there can be a so-called H-bridge 16 in the middle.

Furthermore, the high voltage rail 12 can be connected with the other rail 14 by means of four branches, 18, 20, 22 and 24.

Each branch 18, 20, 22, 24 has a node with the H-bridge main branch 16, denoted as node 18a, 20a, 22a and 24a.

In branch 18, there can be a switch S5 in the branch segment coming from the high voltage rail 12 and before node 18a.

After node 18a, there can be a further switch S₆ in the direction of branch 18, arranged between node 18a and the other rail 14.

In branch 20 there can be a first current source I₁, being arranged between the high voltage rail 12 and a first switch S₁ in branch 20.

After switch S₁ there can be node 20a, followed by a second switch S₂, which is arranged between node 20a and the other rail 14.

Between switch S₂ and rail 14, there can be a second current source I₂.

In branch 22 there can be another current source I₃, which is arranged between the high voltage rail 12 and a switch S₃.

The switch S₃ can be arranged between node 22a and current source I₃.

After node 22a there can be a further switch S₄ in the direction of branch 22, which is arranged between the node 22a and a third current source I₄, which is arranged between switch S₄ and rail 14.

In branch 24, there can be a switch S₇ between high voltage rail 12 and node 24a.

After node 24a there can be another node 24b, which goes to a further branch 26.

Following branch 24, there can be a further switch S₈ between node 24b and rail 14.

In branch 26, there can be resistors R₁ and R₂ and between resistors R₁ and R₂, there can be a branch 28 going to an analog-digital-converter (ADC).

In branch 16 of the H-bridge and between nodes 20a and 22a, there is a capacitor C_{CAN}.

Branch 16 can be extending over nodes 22a, 20a, 18a vis-à-vis the tissue T and forms a part of the housing CAN, which is in conductive/galvanic contact with the tissue T.

Rail 14 is connected to ground GND.

In the most simple form of a regulated CAN controller circuit, only current source I₃ with its enable switch S₃, I₄ with its enable switch S₄ and switch S₆ are present and all other components are removed except for CAN capacitor C_{CAN} and resistive divider R₁ and R₂ as shown in FIG. 1B.

FIG. 1C depicts the working steps of a process 100, where a regulated CAN controller circuit is intended to work as follows.

Initially, the CAN capacitor C_{CAN} is not charged at all. In step 101, switch S₆ can be closed (made conducting) and the capacitor can be charged up through enabled current source I₃ (switch S₃ is closed, that is, made conducing) to a desired voltage level. The voltage level of the capacitor can be monitored through the (high-ohmic) resistive divider R₁ and R₂ whose (low-voltage) output goes to a digital-to-analogue converter (ADC), for example, available as part of a microcontroller unit (MCU, not shown, also forming the energy controlling element) of an IPG.

Next, in step 102, switch S₆ can be opened (made non-conducting) and (enabled) current source I₃ can be set to a current level so that it enters its (low-ohmic) linear region and as such, the current source acts as a switch itself, connecting the CAN capacitor C_{CAN} to the high voltage rail V_{HIGH} (provided through a boost converter connected to the IPG battery, the energy source of the IPG). If current source I₃, acting as a switch, has an excessive on-resistance, a separate switch S₇ can be connected in parallel to current source I₃ and its switch S₃ and this (very low-ohmic) switch can be closed instead of using current source I₃ (that can be turned off by opening switch S₃ after the CAN capacitor has been charged up to the desired voltage if switch S₇ is added).

In step 103, with the CAN at the desired voltage (e.g., a voltage selected to ensure that all electrode current sources of the output stages of the STIM engine are in their high-ohmic output regime) a stimulation pulse can be generated. For unipolar stimulation through the CAN and a biphasic stimulation current pulse, the CAN capacitor C_{CAN} can be charged up further during one phase of the biphasic stimulation current pulse when the CAN sources current to the tissue with this delivered current to the tissue drawn from the high-voltage supply V_{HIGH}. However, the CAN capacitor C_{CAN} can be discharged again, not taking any charge from the high-voltage supply V_{HIGH}, when the CAN sinks current from the tissue during the other phase of the biphasic stimulation current pulse. In some embodiments, in the case of a perfectly balanced biphasic stimulation current pulse with a zero net charge content, the voltage across the capacitor will be the same as before the stimulation pulse was given.

In step 104, the capacitor voltage may be regulated back to a desired voltage or to within a desired voltage window. Such regulation may be necessary when the capacitor voltage begins to drift away from the desired voltage or outside the desired voltage range (e.g., in response to a charge imbalanced stimulation pulse or pulse train). In some embodiments, during the grounding phase of the stimulation, when no stimulation pulses are present, current source I₃ can be disabled through switch S₃ (making it non-conducting, or S₇ is opened when it was used instead of current source I₃) and switch S₆ is closed again, the CAN capacitor voltage can be measured through the resistive divider R₁ and R₂ as before and the MCU switches on either current source I₃ or I₄ depending on whether the CAN capacitor needs to be charged or discharged, respectively, to regulate it back to the desired voltage, while monitoring the voltage level through the resistive divider. Ideally, the capacitor has its desired voltage again before the end of the grounding phase but if not, the regulation can continue during the next grounding phase, and the next stimulation pulse or pulses can be given as already explained. In the shown embodiment, the monitoring of the voltage across the CAN capacitor C_{CAN} takes place during the grounding phase of the stimulation period when no pulses are output and can be done via branch 26, resistor R1, resistor R2 and the ADC via branch 28. The monitoring, which delivers input to the energy controlling element, makes sure that the housing or housing portion is maintained within and/or adjusted to a desired predefined voltage (range) and/or electrical potential (range) by (or using) the capacitor, the current sources and the energy source.

One non-limiting exemplary variant to the aforementioned theme is that, in case of monophasic stimulation, current source I₁, enabled through its switch S₁, can push the same current through the CAN to the tissue as is taken from it through the monophasic stimulation pulse. In this way, ideally, the CAN voltage does not change, and no time needs to be reserved to regulate the capacitor voltage, enabling higher frequency stimulation as it may relax the minimum required grounding time.

In another non-limiting exemplary variant to the aforementioned them is that, in case of biphasic stimulation, both current source I₁ and current source I₂ can advantageously be used in such a way that the CAN current is not sinked or sourced by the CAN capacitor but by either one of these current sources during the biphasic stimulation pulse, also minimizing the time needed for regulation.

Of course, in the real world, even with the use of current source I₁ and/or I₂, the capacitor voltage must be regulated back to what is desired eventually but this may only be needed after multiple pulses or can be done during multiple grounding phases of short duration enabling higher frequency stimulation.

In yet another non-limiting exemplary embodiment, in case of unipolar stimulation, that is, solely between the stimulation electrodes (not shown) and the CAN, with monophasic pulses, one could close switch S₅ during stimulation and close switch S₆ during grounding while leaving all other switches open as long as this stimulation regime lasts. Any charge accumulated on the CAN capacitor, for example, via leakage current through resistors R₁ and R₂, can be removed during the grounding phase of the stimulation when not only switch S₆ is closed but also switch S₈. In particular, for monophasic, unipolar stimulation, no CAN control is needed at all because the CAN potential can be set without the CAN capacitor.

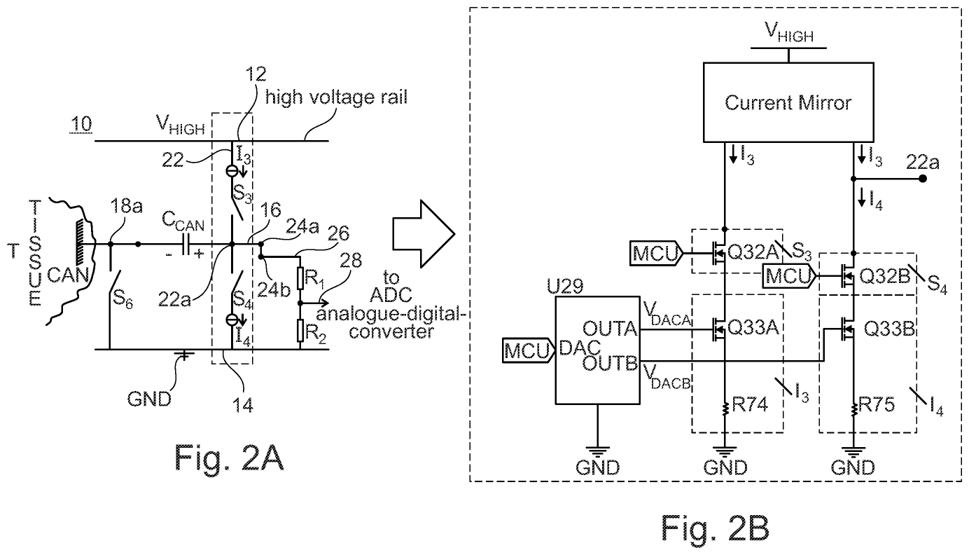

In FIG. 1A, the current sources I₁ and I₂ with their enable switches S₁ and S₂, resp., in the right-hand side branch (branch 22) of the H-bridge, as indicated by the dashed box in FIG. 2A, can be realized with the circuit shown in FIG. 2B: Current source I₃ can be composed of a series stage with NMOS transistor Q32A and resistor R74. Its output current can be determined by output voltage V_{DACA} of output OUTA of (dual) digital-to-analogue converter (DAC) U29. Current source I₄ uses a similar series stage topology of Q33B with R75 connected to output OUTB of the same DAC; The DAC output voltages OUTA and OUTB can be set by a MCU, and consequently, via each series stage, the output current of current source I₃ and I₄, resp.; Current source I₃ and I₄ can be enabled via NMOS transistor switch Q32A and Q32B, resp. These switches can individually be controlled by the same MCU; The output current of current source I₃ can be copied via a current mirror, connected to the high-voltage rail V_{HIGH}, and added to the output current of current source I₄ at node 22a.

All other current sources with their enable switches as shown in FIGS. 1A, 1B, 3, 4 and 5 can be realized in the same way. In these figures, the stand-alone switches S₅ and S₇ can each be realized with a single PMOS transistor, while switches S₆ and S₈ can each be realized with a single NMOS transistor.

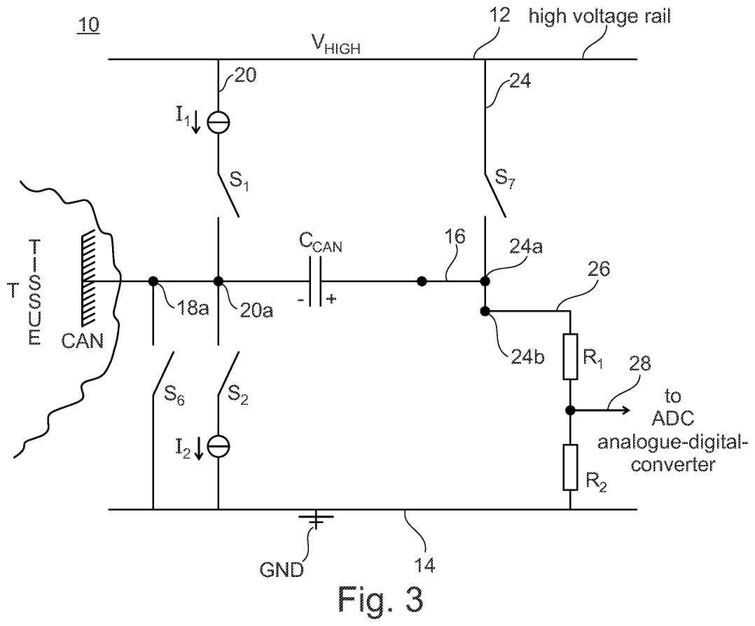

FIG. 1B uses the current sources in the right-hand side branch of the H-bridge to regulate the capacitor voltage during the grounding phases of the stimulation but one can also regulate with only the current sources in the left-hand side branch, branch 20, of the H-bridge as shown in FIG. 3.

For example, if the CAN capacitor voltage is too low, current source I₂ can be enabled, via switch S₂, after the grounding phase of the stimulation. This current charges up the capacitor and during the next grounding phase it can be checked if the capacitor voltage is back to the intended level. If not, the regulation process can be continued, possibly with a further adjustment of the current of current source I₂. The other way around, if the capacitor voltage is too high, current source I₁ with its switch S₁, can be enabled and controlled to regulate the CAN capacitor voltage back to the intended level.

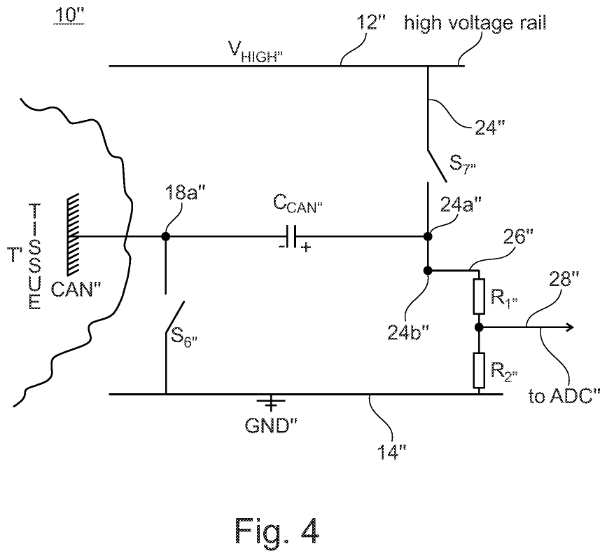

FIG. 4 shows a further embodiment of a control circuit according to the present invention, with which the method according to the present invention may be performed.

The shown embodiment has the same structural and functional features as control circuit 10 as shown in FIG. 1A. Similar or the same features of the control circuit 10 have the same reference number, however with two added apostrophes (″).

The following differences to the embodiment of FIG. 1A exist:

The embodiment of FIG. 4 shows the minimum configuration. The control circuit 10′ can already work with only switch S₆″, capacitor C_{CAN}″, switch S₇″, high voltage rail V_{HIGH}″ and the monitoring element in branch 26″ with its components, R₁″, R₂″, and the connection to analogue-to-digital converter ADC″ via branch 28″ if the stimulation pulses themselves are used to regulate the CAN capacitor voltage.

For example, during the grounding phase of the stimulation, when no stimulation pulses are generated and switch S₆″ can be closed and switch S₇″ can be open, the voltage across the CAN capacitor C_{CAN}″ can be monitored and if it turns out that the capacitor voltage has drifted away from the desired voltage or is outside its desired voltage range, the CAN capacitor voltage can be adjusted after the grounding phase via the stimulation pulses themselves. So, the capacitor voltage regulation does not take place during the grounding phases but during the moments that stimulation pulses are present as was also the case in the embodiment of FIG. 3.

For example, if biphasic stimulation pulses are used for the stimulation and the monitored capacitor voltage turns out to be lower than desired, the MCU adjusts the stimulation pulses (e.g. by changing the amplitude and/or duration of a pulse phase) in such a way that the net delivered charge of the pulses to the tissue becomes negative (temporary negative DC current), and consequently, the CAN capacitor can be charged and its voltage increased after each stimulation pulse. This process can be monitored during each grounding phase and the regulation process can be stopped (by reverting to the original amplitude and/or duration of the impacted pulse phase) once the CAN capacitor voltage has been charged to its intended voltage level.

The other way around, if the monitored capacitor voltage turns out to be higher than desired, the MCU adjusts the stimulation pulses in such a way that the net delivered charge to the tissue becomes positive (temporary positive DC current) until the CAN capacitor voltage has been discharged to its intended voltage level.

This same mechanism to regulate the CAN capacitor voltage via the stimulation pulses themselves can also be used to fine tune the biphasic pulse parameters (e.g. duration of the pulse phases) in such a way that the pulses become (almost) perfectly charge balanced. This minimizes the CAN capacitor voltage drift, and therefore, the amount of regulation actions.

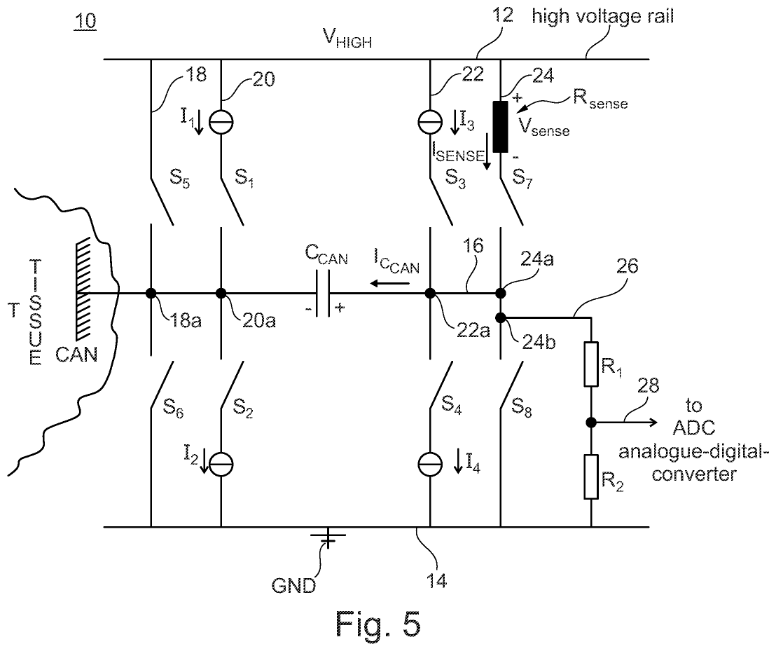

The control circuit of FIG. 1A can be expanded with a second monitoring element that monitors the current flow through the CAN capacitor in between the grounding phases when actual stimulation takes place. This is shown in FIG. 5 where the current monitoring element can be implemented with a (precise) sensing resistor Rₛₑₙₛₑ in between high-voltage rail V_{HIGH} and switch S₇. When switch S₇ is closed, during the stimulation phases of the stimulation, the current Iₛₑₙₛₑ through the sensing resistor Rₛₑₙₛₑ equals the current I_{CCAN} absorbed from or delivered to the CAN by the CAN capacitor. The resulting sense voltage Vₛₑₙₛₑ=Rₛₑₙₛₑ I_{CCAN} can be amplified, digitized and analysed by the MCU for follow-up actions if needed.

For example, in the embodiment of FIG. 1A, for unipolar stimulation, the current of each phase of each stimulation pulse flows through the sensing resistor and this current can be analysed by the MCU. If the current level of one or more phases is out of the specified range, the MCU can try to adjust the involved pulse phase current and if that fails, the stimulation can be stopped and it can be flagged which current sources of which output stages of the stimulation engine are faulty.

The advantage of this second monitoring element is that only a single monitor is needed to monitor all (e.g. 16) output stages of the stimulation engine instead of incorporating a monitoring element in each individual output stage. In this way, a power-efficient and area and volume friendly monitoring function can be realized.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Words such as “and” or “or” mean “and/or” unless specifically directed otherwise. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A neurostimulation system, comprising:
an energy source;
a housing or a housing portion;
a stimulation node;
a stimulation surface for contacting a tissue of a patient; and
a control circuit configured to maintain at least one of the housing or housing portion, the stimulation node, or the stimulation surface at:
a predefined voltage, or
within a predefined voltage range.

2. The system of claim 1, wherein the control circuit comprises at least one energy controlling element that draws energy from or returns energy to the energy source to keep at least one of:
the housing or the housing portion,
the stimulation node, or
the stimulation surface
at a predefined voltage or kept within a predefined voltage range.

3. The system of claim 1, wherein the stimulation surface is maintained at the predefined voltage or within the predefined voltage range using a capacitor.

4. The system of claim 3, further comprising a monitoring element.

5. The system of claim 4, wherein:
the energy source comprises a power source; and
the monitoring element is configured to monitor at least one of:
a voltage across the capacitor;
a voltage of at least one of the housing or the housing portion;
a voltage of the stimulation node;
a voltage of the stimulation surface;
a current through at least one of
the capacitor,
the housing,
the housing portion,
the stimulation node, or
the stimulation surface; or
at least one of a voltage, a current, or a power of the power source.

6. The system of claim 1, wherein the control circuit comprises at least one of
a current source;
a switch;
a voltage source;
a ground; or
a bridge circuit.

7. The system of claim 6, wherein at least one of
the housing or housing portion,
the stimulation node, or
the stimulation surface,
is part of a bridge circuit.

8. The system of claim 7, wherein the bridge circuit comprises an H-bridge circuit.

9. The system of claim 1, wherein the control circuit has a supply voltage within a range from more than 0 V to about 50 V, whereas the supply voltage is supplied by a voltage supply.

10. The system of claim 9, wherein the supply voltage is from about 5 V to about 30 V.

11. The system of claim 9, wherein the supply voltage is from about 20 V to about 30 V.

12. The system of claim 1, wherein the control circuit comprises at least one of:
a capacitor;
a monitoring element;
a switch being arranged in a branch connecting the capacitor with a voltage supply or another voltage source; and
a further switch being arranged in a branch connecting the capacitor with another rail.

13. The system of claim 12, wherein the control circuit comprises at least one further current source.

14. The system of claim 13, wherein the control circuit further comprises a first control switch and a second control switch connected in series.

15. The system of claim 14,
wherein the at least one further current source comprises a first current source and a second current source; and
wherein the first control switch and the second control switch are arranged between the first current source and the second current source.

16. The system of claim 1, further comprising a switch for connecting, to at least one of ground or another rail, at least one of
a capacitor, or
the housing or housing portion.

16. The system of claim 1,
wherein the control circuit is directly or indirectly connected to a stimulation generation module of an implantable pulse generator, and
wherein the stimulation generation module is powered from a single energy source.

17. The system of claim 1, whereas at least one stimulation current source or output stage of the stimulation generation module, during stimulation, remains in at least one of
its high-ohmic output range, or
its compliance region.
